(19) United States
(12) Patent Application Publication
(10) Pub. No.: US 2007/0131227 A1
(43) Pub. Date: Jun. 14, 2007

(54) Aquatic headgear

(76) Inventor: Troy L. Wheelwright, Haverhill, MA (US)

Correspondence Address: BOURQUE & ASSOCIATES;INTELLECTUAL PROPERTY ATTORNEYS, P.A., 835 HANOVER STREET, SUITE 301, MANCHESTER, NH, 03104, US

(21) Appl. No.: 11/387,141

(22) Filed: Mar. 22, 2006

Related U.S. Application Data
(60) Provisional application No. 60/748,711, filed on Dec. 9, 2005.

(51) Int. Cl. A62B 17/04; A61M 16/00; B63C 11/02; A61M 11/00

(52) U.S. Cl. 128/201.22; 128/200.26; 128/200.29; 128/200.14

(57) ABSTRACT

An improved aquatic device for use with goggles features a support base sized and shaped to fit near the back of a user's head and is secured against the user's head by way of the goggle strap. Various equipment may be secured to the support base including a snorkel and/or a light. In the preferred embodiment, the support base includes a connection system that allows the various equipment to be removably secured to the support base such that the user to select the equipment most suited to the intended application. The snorkel preferably includes a splash guard that prevent water from entering the air passageway. Additionally, flexible tubes connect the snorkel secured to the support base to the mouthpiece. The flexible tubes are arranged such that they extend behind the user's head and beneath the user's jaw. As a result, drag is significantly reduced and mobility and comfort are increased.

### CROSS-REFERENCE TO RELATED APPLICATIONS

This application claims the benefit of U.S. Provisional Application Ser. No. 60/748,711 for HEAD MOUNTED FLEXIBLE SNORKLE FOR SWIMMERS AND DIVERS filed Dec. 9, 2005.

### TECHNICAL FIELD

The present invention relates to snorkeling and scuba diving and more particularly, relates to a head mounted aquatic system.

### BACKGROUND INFORMATION

It can be appreciated that swimming snorkels have been in use for years. Traditionally, swimming snorkels are comprised of a semi-hard rubber or plastic tube that is used by divers or snorkelers for breathing while swimming face down in the water. The existing products usually have a molded rubber portion attached to the lower end of the snorkel tube that is angular in shape for directing the mouthpiece to the divers mouth. The snorkel tube is usually secured to the goggle strap on one side of the head by a rubber connector or clip. Some devices found in the market place have improvement features that include a purge valve located next to the mouthpiece for purging excess water from the breathing tube. Also, some units incorporate a valve mechanism on the top of the snorkel which helps prevent water from entering while breathing. Some existing units have a snorkel tube that is shaped so that they more easily form to the side of the divers head.

While these traditional snorkels are generally effective, they suffer from numerous limitations and disadvantages. For example, conventional swimming snorkels are generally awkward and uncomfortable to use and difficult to adjust. Water flowing past the swimmer's head exerts a lot of force against the snorkel. Because the snorkel is secured to the goggle strap at a single point on the goggle strap, much of this force is transmitted by the snorkel to the goggle strap thereby causing discomfort for the user. Additionally, the proximity of the snorkel to the goggles can make fitting and sealing the goggles to the user's head difficult and can limit the mobility of the user. Moreover, when the snorkel is not in use, the hard tube hangs like a weight on the side of the divers goggles causing further discomfort and flops around thereby getting in the way. As a result, even though most scuba/snorkeling organizations suggest wearing a snorkel while scuba diving, many scuba divers remove their snorkels.

Another limitation of the known snorkels is that it is often difficult to identify one individual from another. For safety reasons, divers and snorkelers generally team up with at least one swim “buddy”. It is the responsibility of each swim buddy to watch over the other and come to his/her aid. Swimmers commonly wear wet suits (often having hoods which cover the user's head) to protect against the cold and these wet suits often look very similar. As a result, identifying a person's swim buddy from the others while underwater is often very difficult and can lead to a very dangerous situation if the swim buddies become separated.

Yet another limitation of the known snorkel and diving systems is that they are difficult to use at night. When diving at night, it is necessary to have a source of light to illuminate the diver's surroundings. As anyone who has dove can attest, diver's are generally required to carry numerous other pieces of equipment, much of which must be held in order to use. The known light sources for diving are typically waterproof flashlights. The user must generally hold the flashlight at all times, thereby limiting the diver's ability to utilize other pieces of equipment.

Accordingly, there exists a need for an improved aquatic device for swimmers and divers that will overcome the shortcomings of the prior art devices described above. The improved aquatic device should preferably eliminate the need to secure the snorkel to the side of the strap of the goggle on the side of the user's head. Additionally, the improved aquatic device should preferably position the snorkel on the back of the head instead of on the side of the face, thereby making the snorkel more comfortable and freeing up space around the mask and face area making it easier to adjust the mask for a leak free fit. The improved aquatic device should also preferably eliminate the hard plastic or rubber snorkel tube that extends up the side of the face as used in known designs thereby increasing the user's comfort.

There also exists a need for an improved aquatic device that eliminates the rigid tubing used in the known snorkels, hereby further increasing the user's comfort and mobility. The improved snorkel system should preferably prevent or reduce the tendency for the snorkel to pull on the goggle strap and cause discomfort. There also exists a need for an improved snorkel system that can be permanently secured to the back of a divers hood for use by scuba divers in cold water environments.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention, which is not to be limited except by the following claims.

### SUMMARY

According to one embodiment, the present invention features an aquatic device comprising a support base sized and shaped to substantially fit only over a posterior superior portion of a user's head and means for connecting the support base to a goggle strap. The support base may be generally flexible or generally rigid, and optionally includes a layer of a non-slip material (such as neoprene) along the interior surface.

The goggle strap may include a first and a second separate strap permanently secured to a first and a second generally opposite side of the support base, respectively. Alternatively, at least one channel may be disposed within the support base that is sized and shaped to accept at least a portion of the goggle strap such that the goggle strap passes through at least one a portion of the support base.

The aquatic device may also feature a snorkel secured to and extending generally outwardly and away from the support base and at least one flexible tube. The flexible tube has a first end connected to an air passage of the snorkel and a second end connected to a mouthpiece. The air passageway of snorkel optionally includes a first aperture disposed proximate a distal end of the snorkel and at least a second aperture fluidly connected on opposite ends of the air passageway, wherein the at least a second aperture is disposed proximate the support base. The second aperture is preferably disposed proximate a back region of the support base such that when the aquatic device is worn on the user's head, the second aperture generally faces towards a neck of the user's head. The flexible tube is preferably sized and shaped such that when the aquatic device is worn on the user's head, the flexible tube is substantially disposed behind the user's head, around a chin of the user, and connects with the mouth piece. The aquatic device may feature a first and a second flexible tube adapted to be disposed on a first and a second generally opposite side of the user's head when worn.

The support base may optionally include means for removably securing the snorkel to the support base. For example, the support base may be removably secured to snorkel by way of a channel (preferably disposed within the support base) and a pin sized and shaped to fit within the channel. Optionally, a biased tap is disposed proximate an open end of the at least one channel and urges the snorkel within the channel.

The snorkel may also include a splashguard. The splashguard preferably includes a flanged region disposed proximate a distal of the air passageway of the snorkel and a buoyant float moveably disposed about an exterior portion of the snorkel. When the buoyant float is above water, gravity moves the buoyant float to a first position such that the air passageway is substantially unobstructed. When the buoyant float is below water, gravity moves the buoyant float to a second position such that the buoyant float engages the flanged region and the air passageway is substantially sealed.

The aquatic device may optionally includes at least one light. A power source may be located proximate the support base or remotely from the support base. The light is preferably removably connected to the support base, though it may also be permanently secured to the support base.

According to another embodiment, the present invention features a sport apparatus. The sport apparatus includes a goggle, a strap secured to the goggle, a support base secured to the strap and sized and shaped to substantially fit only over a posterior superior portion of a user's head, and engagement means for removably securing an object to the support base. The object preferably includes and snorkel and/or a light.

### BRIEF DESCRIPTION OF THE DRAWINGS

These and other features and advantages of the present invention will be better understood by reading the following detailed description, taken together with the drawings wherein:

FIG. 1 is a side plan view of one embodiment of the improved aquatic device according the present invention;

FIG. 2 is a top plan view of one embodiment of the support base of the improved aquatic device according to the present invention;

FIG. 3 is a top plan view of another embodiment of the support base of the improved aquatic device according to the present invention;

FIG. 4 is a top, front plan view of one embodiment of the support base of the improved aquatic device in conjunction with a snorkel according to the present invention;

FIG. 5 is a top, back plan view of one embodiment of the support base of the improved aquatic device in conjunction with a snorkel having a splash guard according to the present invention;

FIG. 6 is a perspective rear view of one embodiment of the mouthpiece of the improved aquatic device according to the present invention;

FIG. 7 is a top plan view of one embodiment of the support base of the improved aquatic device having a connection system according to the present invention;

FIG. 8 is a front plan view of the support base of the improved aquatic device having a connection system shown in FIG. 7 according to the present invention;

FIG. 9 is a top, front plan view of one embodiment of the support base of the improved aquatic device and a removable snorkel according to the present invention;

FIG. 10 is a front plan view of one embodiment of the removable snorkel of the improved aquatic device according to the present invention; and

FIG. 11 is a front view of one embodiment of the removable light of the improved aquatic device according to the present invention.

### DETAILED DESCRIPTION OF THE PREFERRED EMBODIMENTS

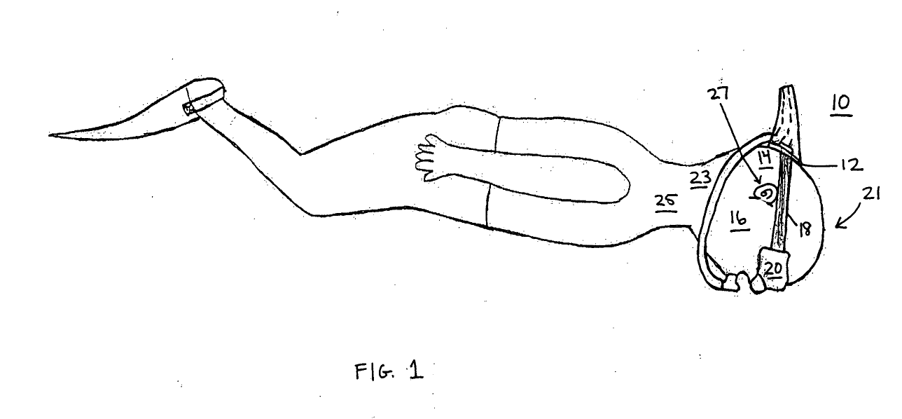

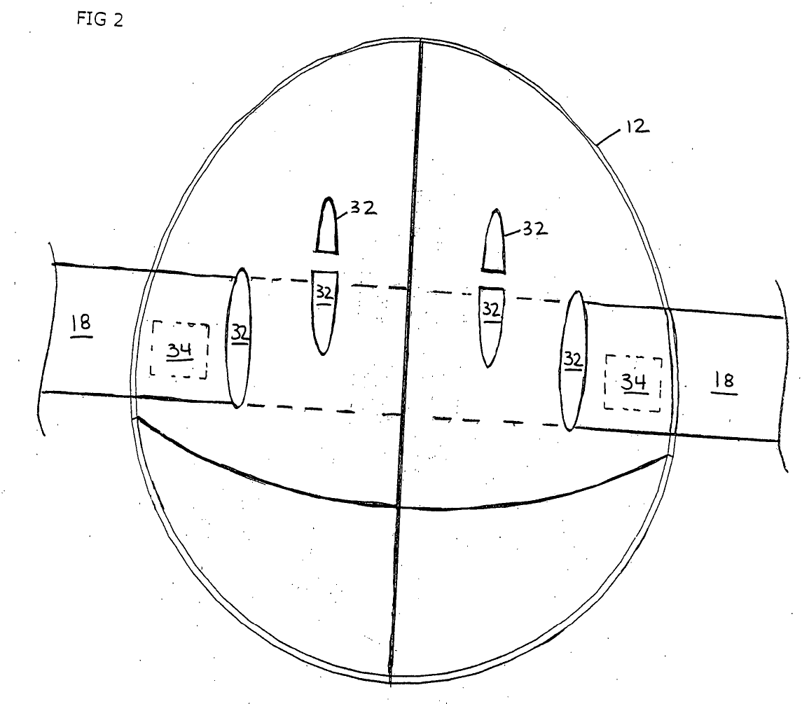

According to one embodiment, the present invention features an improved aquatic device 10, FIG. 1, for use with snorkeling or scuba diving that features a support base 12 adapted to disposed proximate the back region 14 of a user's head 16 which is held in place by way of a strap 18 of a pair of goggles 20. Referring specifically to FIGS. 2-7, the support base 12 has a shape contoured to fit over at least a portion of the back 14 (FIG. 1) of a user's head 16 and preferably conforms to the user's head 16. As will be described in greater detail hereinafter, locating the support base 12 proximate the back of the user's head 16 facilitates the use of a snorkel 22 (FIG. 1) and/or other devices, especially when the user's head 16 is face down in the water.

In the exemplary embodiment, the support base 12 has a generally dome-like shape that extends over the posterior superior portion of the user's head 16 such that the support base 12 cradles the user's head 16. The support base 12 is not intended to serve as a protective gear, but rather it is important to note that the support base 12 should be sized and shaped to facilitate movement and/or range of motion of the user's head 16. As a result, the support base 12 preferably does not extend over a top region 21 (FIG. 1) of the user's head 16, over the joint 23 between the user's head 16 and neck 25, or over the user's ears 27. The support base 12 is preferably large such that it substantially does not move relative to the user's head 16 during use, though it may be enlarged to create a pleasing aesthetic appearance. The support base 12 may, however, be incorporated into a diving hood, however, the placement of the support base 12 is preferably disposed proximate posterior superior portion of the user's head 16.

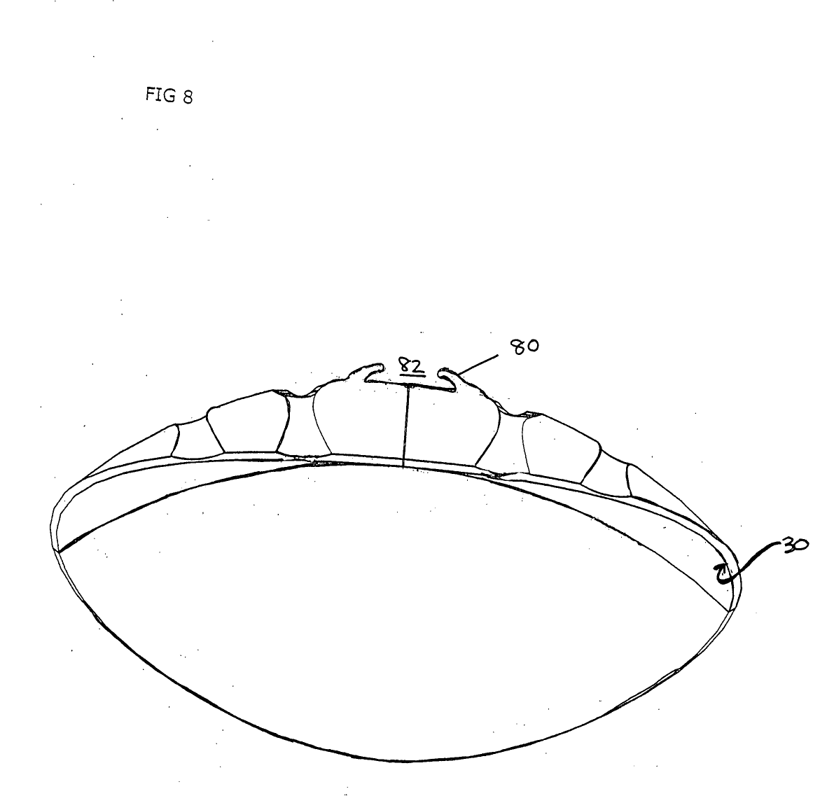

In the preferred embodiment, the support base 12 may be constructed from a flexible, resilient material such as, but not limited to, plastic, neoprene, rubber, or any other suitable material. Alternatively, the support base 12 may be constructed from a generally rigid material. Optionally, a lower or interior surface 30 (best seen in FIG. 8) may include a non-slip, high-friction and/or cushioning surface such as, but not limited to, silicon rubber or neoprene. The non-slip and/or cushioning surface further enhances the user's comfort and reduces the likelihood of the support base 12 moving relative to the user's head 16.

As discussed above, the support base 12 is held in place by means of the straps 18 of the goggles 20. According to one embodiment, the strap 18, FIG. 2, may be secured to the support base 12 by means of one or more apertures, channels, or slots 32 sized and shaped to accept at least a portion of the strap 18. The apertures 32 may be located in various regions of the support base 12 to allow the strap 18 to be secured to various areas of the support base 12. This also allows the location of the support base 12 with respect to the user's head 16 to be slightly altered to better fit the contours of the user's head 16, thereby increasing the user's comfort.

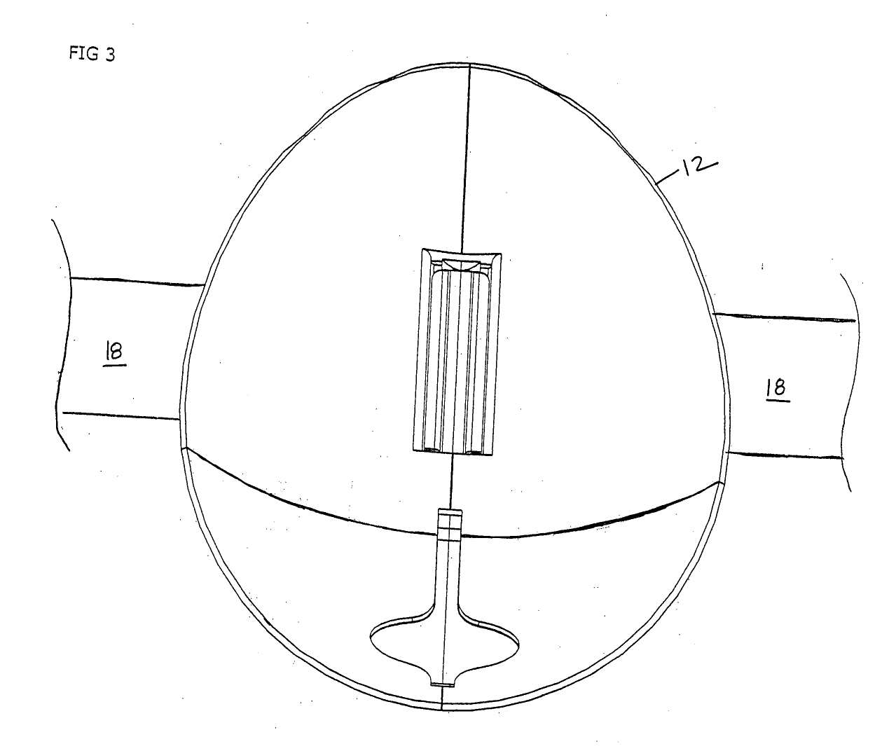

The strap 18 may also be secured to the support base 12 by other methods. For example, the strap 18 and the base 12 may feature one or more hook and loop type fasteners, buttons, or the like 34. Alternatively, the strap 18, FIG. 3, may be secured substantially permanently to the support base 12 using an adhesive, welding, mechanical clamp, or the like.

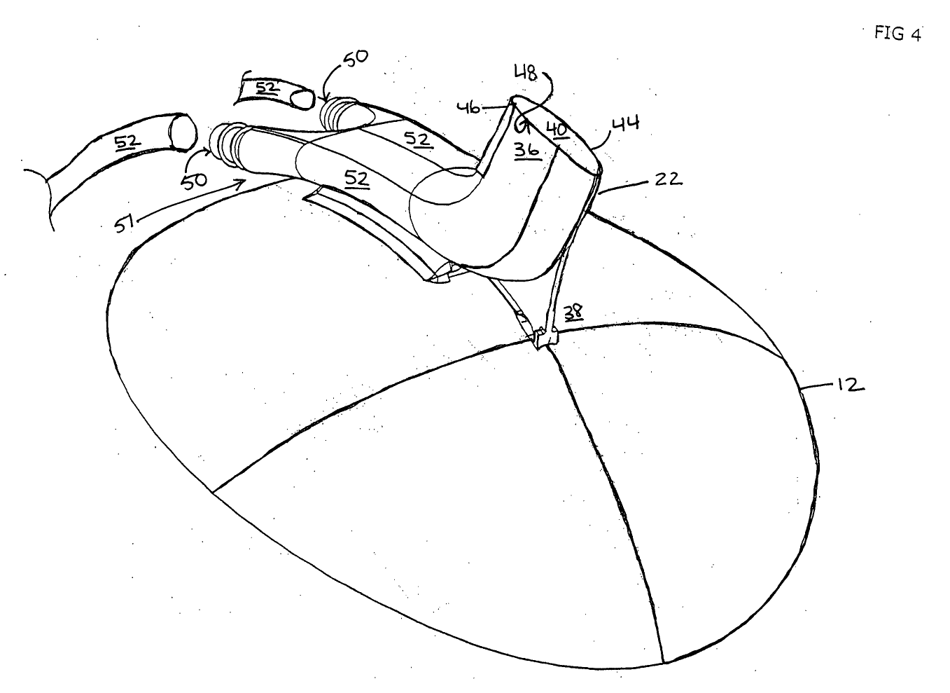

According to one embodiment, the improved aquatic device 10, FIG. 1, optionally includes a snorkel 22. In a first embodiment, the snorkel 22, FIG. 4, may be permanently secured to (or an integral unit with) the support base 12. The snorkel 22 preferably includes an elongated region 36 extending generally upwards and away from a top surface 38 of the support base 12. The exact angle which the snorkel 22 extends outwardly from the support base 12 will depend on the intended location of the support base 12 and the snorkel 22 as well as the overall dimensions of the snorkel 22, and is within the knowledge of one of ordinary skill in the art. For illustrative purposes only, the snorkel 22 preferably extends outwardly approximately 3.5 inches and is substantially tangential to the top surface 38 of the support base 12.

The snorkel 22 also preferably includes at least one air inlet 40, air outlet 50, and passageway 52 fluidly connecting the inlets and outlets 40, 50. The terms “inlet” and “outlet” are intended to denote the flow of air through the snorkel 22 during inhalation; those skilled in the art will readily recognize that during exhalation the airflow will change. The snorkel 22 may additionally include separate passageways 52 for inhalation and exhalation, each having dedicated air inlets 40 and air outlets 50 based on the flow of air.

The air let 40 may encompass any of the various known designs of air inlets 40 and the present invention is not limited to any one particular design of an air inlet 40 unless specially claimed as such. For illustrative purposes only, the air inlet 40 may simply feature an aperture 44 disposed proximate the distal end 46 on the top region 48 of the snorkel 22. Alternatively, the air inlet 40 may be disposed on the side and/or the back of the snorkel 22. The air inlet 40 may further include any of the known splash protectors or other devices to prevent/reduce water from being drawn into the snorkel 22.

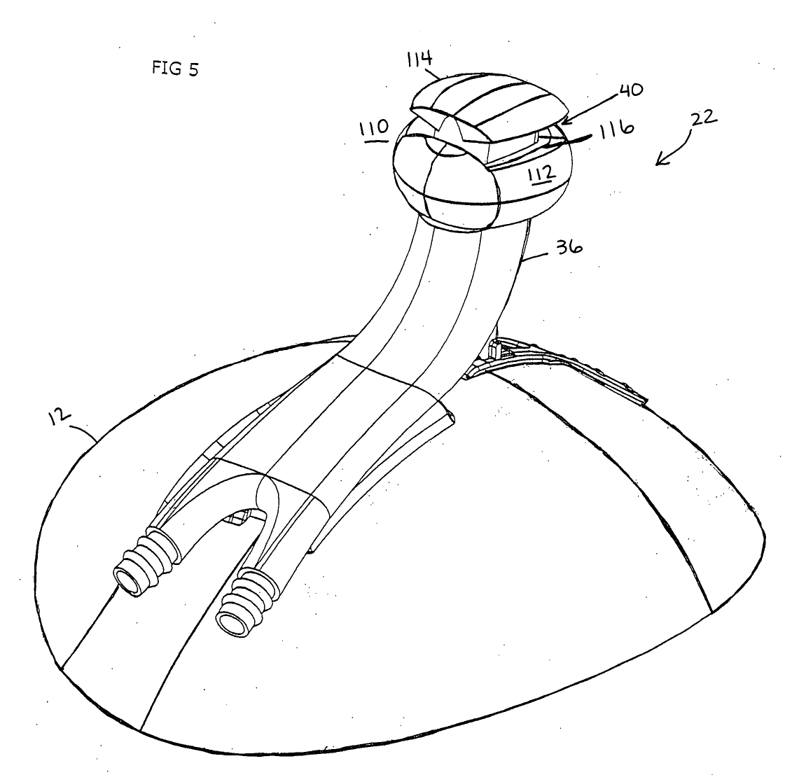

For example, the snorkel 22, FIG. 5, may feature a novel splash guard 110 that reduces the possibility of water entering the air inlet 40. The splash guard 110 preferably includes a buoyant float 112 that moves along a region of the elongated portion of the snorkel 22 and a flanged region 114. The buoyant float 112 preferably features a inversed flange region having a perimeter that substantially corresponds to at least a portion of the flanged region 114. While the air inlet 40 is above water, the gravity causes the float 112 to move downward away from the flanged region 114, thereby opening the air inlet 40 to the atmosphere. When submerged, the float 112 moves upward against the flanged region 114, thereby sealing the air inlet 40 along at least the perimeter of the buoyant float 112 and preventing water from entering the passageway 52. The float 112 and/or the flanged region 114 may include a seal 116 such as, but not limited to, an o-ring type seal to further prevent water from entering the passageway 52.

In the preferred embodiment, the air outlet 50 are disposed proximate the support base 12, preferably proximate a back or lower region 51 of the support base 12 such that when the improved aquatic device 10 is worn on the user's head 16, the air outlets 50 are generally facing towards the neck 25 of the user's head 16. One or more flexible tubes 52, FIGS. 1 and 4, are connected (preferably removably connected) to the air outlets 50 and to the mouthpiece 54 (FIG. 1). The flexible tubes 52 may also feature inline connectors disposed a distance away from either the mouthpiece 54 or the air outlets 50 to facilitate removal or installation of the improved aquatic device 10 on the user. As best shown in FIG. 1, the flexible tubes 52 are preferably of a sufficient length such when the mouth piece 54 is disposed in the user's mouth, the flexible tubes 52 go behind the user's head 16, around the user's chin, and connect with the mouth piece 54. Having the flexible tube 52 disposed in this manner increases the overall flexibility and comfort of the improved aquatic device 10 since nothing is disposed against the user's ear 27 and significantly reduces the likelihood of the snorkel being in the way during use or getting caught on something.

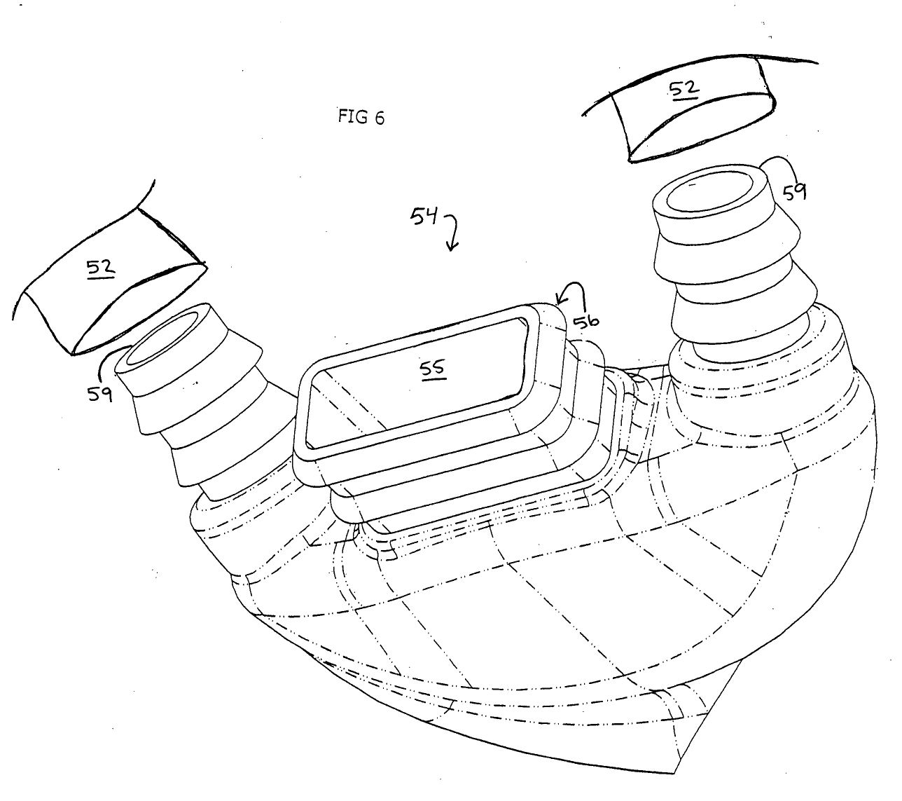

In the exemplary embodiment, the improved aquatic device 10 features a first and a second flexible tube 52 wherein each of the flexible tubes 52 is disposed on opposite sides of the user's head 16 (i.e., on the left and right side of the user's head 16) and connects to the mouthpiece 54. Referring specifically to FIG. 6, the mouthpiece 54 features a flanged region 56 having an aperture 55 sized and shaped to fit within the user's mouth as is well known. The mouthpiece 54 also preferably features at least one passageway fluidly connecting the aperture 55 of the flanged region 56 to at least one opening 59 which is sized and shaped to be secured to the flexible tubes 52, thereby forming an overall passageway between the aperture 55 in the mouthpiece 54 and the air inlet 40 of the snorkel 22. The opening 59 preferably features a flanged region and/or a connector such that the flexible tubes 52 can be removably secured/connected to the mouthpiece 54. Alternatively, the flexible tube 52 may be permanently secured to the mouthpiece 54. The mouthpiece 54 also preferably includes a purge valve (not shown) as is well known to those skilled in the art.

This embodiment further increases the comfort in numerous ways. For example, the user of two flexible hoses 52 equalizes the drag on both sides of the user's head 16 and also reduces the overall drag since the flexible hoses 52 are generally out of the flow of the water since most of the flexible tubes are beneath the user's head 16 unlike the known snorkels which along the side of the user's head and therefore directly in the flow of the water. Additionally, having two flexible tubes 52 allows the mouthpiece to hang close to the user's mouth when not in use (in contrast to the known snorkel designs in which the mouthpiece dangles about one side of the user's head 16) and allows the diameters of each flexible tube to be reduced while still maintaining a comparable overall airflow rate and pressure drop. Moreover, the use of two separate flexible tubes is particularly suited to applications having separate inhalation and exhalation passageways as described above.

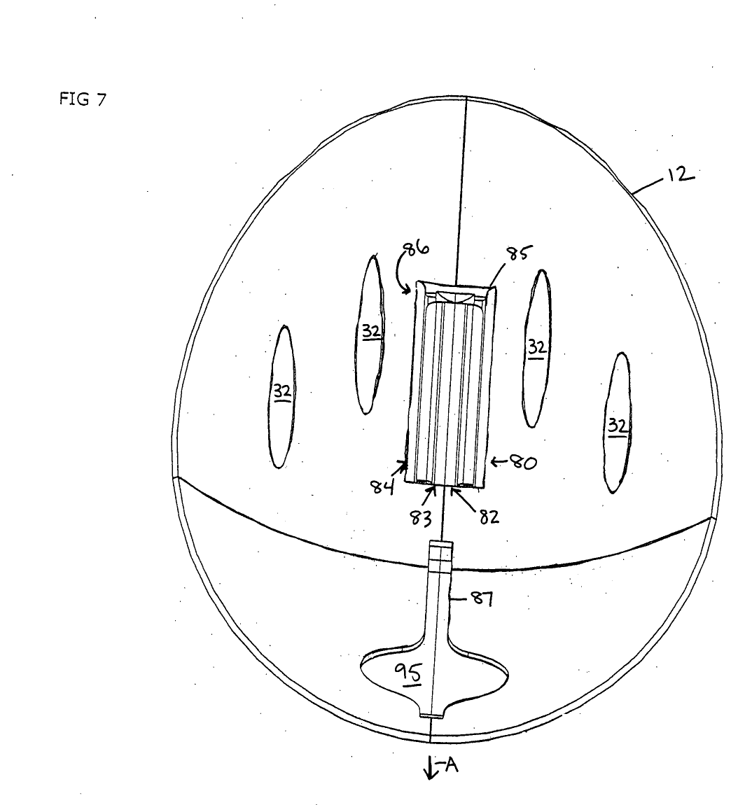

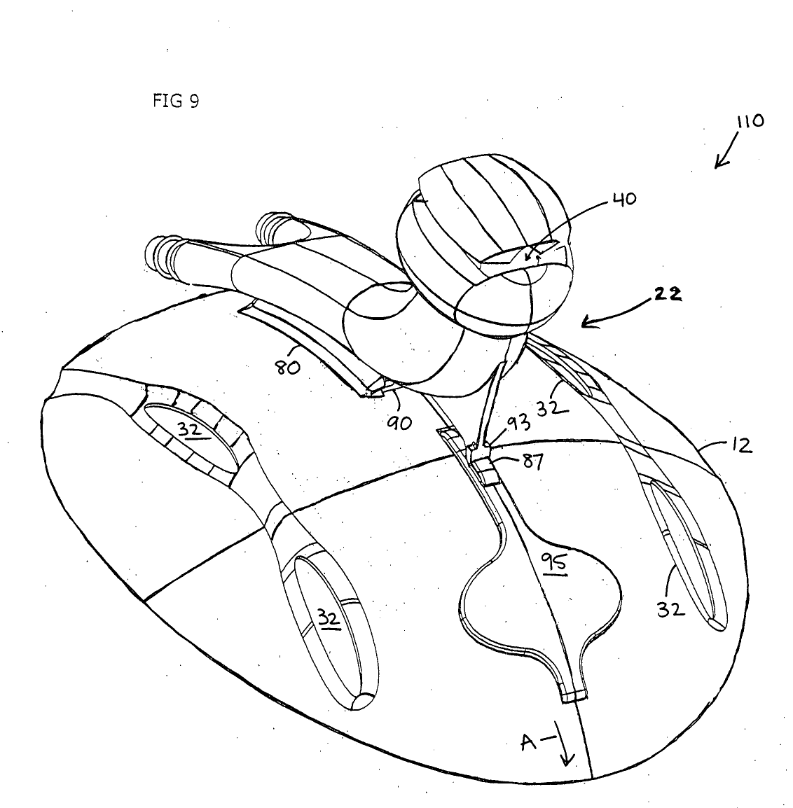

According to a preferred embodiment, the support base 12, FIGS. 7 and 8, of the improved aquatic device 10 optionally features at least one connection system 80 that allows various equipment to be secured to the support base 12 as will be explained in greater detail hereinbelow. For illustrative purposes only, the connection system will be explained in conjunction with a snorkel 22 (FIG. 9), though this is not intended to be a limited of the present invention unless otherwise specifically claimed as such. It is important to note that the improved aquatic device 10 may feature multiple connection systems 80 disposed about the support base 12 such that two or more pieces of equipment (such as, but not limited to, a snorkel 22 and/or a light 100) may simultaneously be connected to the support base 12.

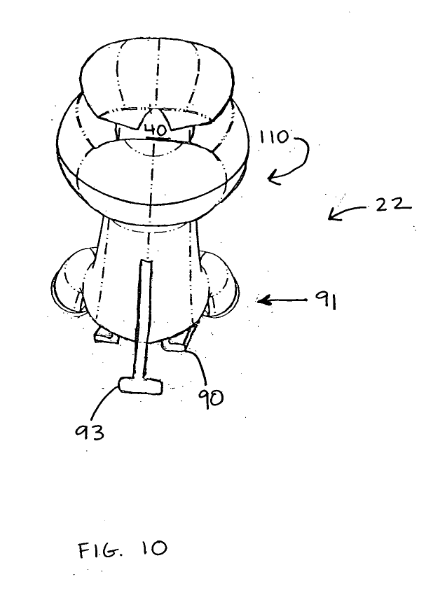

The connection system 80 may feature any device known to those skilled in the art such as, but not limited to, various fasteners including a hook and loop type fasteners, magnetic fasteners, suction fasteners, clamp fasteners, and other known mechanical fasteners. In the preferred embodiment, the connection system 80 features at least one channel, groove, or slot 82 (best seen in FIG. 8) such as, but not limited to, a dovetail type slot, having an opening 83 disposed proximate a first end 84 of the channel 82 and a flanged or stopper 85 disposed proximate a second end 86 of the channel 82. The various equipment (such as the snorkel 22, FIG. 10), preferably feature a protrusion or pin 90, FIGS. 9 and 10) disposed proximate the base 91 of the snorkel 22 sized and shaped to fit within the groove 82 of the connection system 80 in the support base 12. Optionally, a biased tab 87 (FIGS. 7 and 9) may be used to secure/lock the various equipment within the channel 82 by applying a force against the base 91 of the snorkel 22 urging the snorkel 22 against the flanged region 85 of the channel 82. The base 91 of the snorkel 22 may feature a protrusion 93 disposed proximate the first end 82 of the channel 82 that the bias tab 87 applies the force against. The bias tab 87 may optionally feature an enlarged region 95 to facilitate moving the biased tab 87 in the direction of arrow A in order to release the biased tab 87. This enlarged region 95 is particularly advantageous since divers often wear thick gloves to protect their hands.

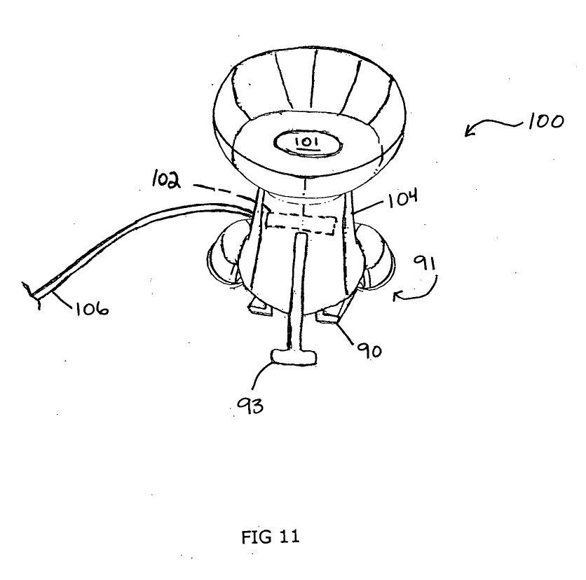

According to another embodiment, the improved aquatic device 10 may feature a light 100, FIG. 11, that may be secured to the support base 12 using any of the connection systems 80 described above. Alternatively, the light 100 may be permanently secured to the support base 12 such that the light 100 is an integral piece of the support base 12. The light 100 preferably includes a light source 101 (such as, but not limited to, an LED or traditional bulb) and a power source 102 (such as a battery or the like). The power source 102 may be disposed within the body 104 of the light 100. Alternatively, the power source 102 may be disposed remotely and connected to the light 100 by one or more wires 106. For illustrative purposes only, the power source 102 may be secured to the dive tanks, buoyancy-compensators device (BV), and/or other gauges.

The use of a light 100 secured to the support base 12 of the present invention eliminates the requirement of the user having to hold a light in their hand, thereby freeing one of the user's hands and eliminating the possibility of the user accidentally dropping the light. Moreover, because the light 100 is secured to the user's head 16, the light 100 will move with the user's head 16 ensuring that the light 100 is always pointing in the direction that the user is looking.

The light 100 may also feature a light (such as a 360 degree light) used primarily for identification purposes. As discussed above, identifying an individual while in the water is often difficult because of the reduced visibility and ability to communication as well as common, non-descript wetsuits. The light 100 may feature multiple light sources 101 in various colors and which blink in various patterns. The use of various colors and blinking patterns allows users to quickly identify a specific individual from others, and also facilitates finding the individual while under water. While the snorkel 22 and the light 100 have been described separately, this is not a limitation of the present invention and the two may be combined.

As mentioned above, the present invention is not intended to be limited to a system or method which must satisfy one or more of any stated or implied object or feature of the invention and should not be limited to the preferred, exemplary, or primary embodiment(s) described herein. The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as is suited to the particular use contemplated. All such modifications and variations are within the scope of the invention as determined by the claims when interpreted in accordance with breadth to which they are fairly, legally and equitably entitled.

The invention claimed is:

1. An aquatic device comprising:
a support base sized and shaped to substantially fit only over a posterior superior portion of a user's head; and
means for connecting said support base to a goggle strap.

2. The aquatic device as claimed in claim 1 wherein said support base is generally flexible.

3. The aquatic device as claimed in claim 2 wherein said support base includes a substantially concaved shape and a layer of a non-slip material disposed about an exterior surface within said interior region.

4. The aquatic device as claimed in claim 3 wherein said non-slip material includes neoprene.

5. The aquatic device as claimed in claim 1 wherein said means for connecting said support base to said goggle strap includes a first and a second separate goggle strap permanently secured to a first and a second generally opposite side of said support base, respectively.

6. The aquatic device as claimed in claim 1 wherein said means for connecting said support base to said goggle strap includes at least one channel disposed within said support base sized and shaped to accept at least a portion of said goggle strap, wherein said goggle strap passes through at least a portion of said support base.

7. The aquatic device as claimed in claim 1 further including a snorkel secured to and extending generally outwardly and away from said support base, said snorkel having at least one air passage.

8. The aquatic device as claimed in claim 7 further including at least one flexible tube, said flexible tube having a first end connected to said air passage of said snorkel and a second end connected to a mouthpiece.

9. The aquatic device as claimed in claim 8 wherein said air passageway of snorkel include a first aperture disposed proximate a distal end of said snorkel and a second aperture fluidly connected about an opposite end of said air passageway, wherein said second aperture is disposed proximate said support base.

10. The aquatic device as claimed in claim 9 wherein said second aperture is disposed proximate a back region of said support base such that when said aquatic device is worn on said user's head, said second aperture generally faces towards a neck of said user's head.

11. The aquatic device as claimed in claim 9 wherein said flexible tube is sized and shaped such that when said aquatic device is worn on said user's head, said flexible tube is substantially disposed behind said user's head, around a chin of said user, and connects with said mouth piece.

12. The aquatic device as claimed in claim 11 wherein said flexible tube further includes a first and a second flexible tube adapted to be disposed on a first and a second generally opposite side of said user's head, respectively.

13. The aquatic device as claimed in claim 7 wherein said support base further includes means for removably securing said snorkel to said support base.

14. The aquatic device as claimed in claim 13 wherein said means for removably securing further includes at least one channel and at least one pin sized and shaped to fit within said at least one channel.

15. The aquatic device as claimed in claim 14 wherein said at least one channel is disposed within said support base.

16. The aquatic device as claimed in claim 15 further including a biased tap disposed proximate an open end of said at least one channel for biasing said at least one pin towards said at least one channel.

17. The aquatic device as claimed in claim 7 wherein said snorkel includes at least one light.

18. The aquatic device as claimed in claim 7 wherein said snorkel includes a splashguard.

19. The aquatic device as claimed in claim 18 wherein said splashguard includes:
a flanged region disposed proximate a distal of said air passageway; and
a buoyant float moveably disposed about an exterior portion of said snorkel such that when said buoyant float is above water, gravity urges said buoyant float towards a first position wherein said air passageway is substantially unobstructed, and when said buoyant float is below water, gravity urges said buoyant float towards a second position wherein said buoyant float engages said flanged region and said air passageway is substantially sealed.

20. The aquatic device as claimed in claim 1 wherein said support base includes at least one light.

21. The aquatic device as claimed in claim 20 further including a power source secured to said support base and electrically connected to said at least one light.

22. The aquatic device as claimed in claim 20 further includes a power source remotely located from said support base and said light.

23. The aquatic device as claimed in claim 20 wherein said support base further includes means for removably securing said light to said support base.

24. An aquatic device comprising:
a support base having a substantially concaved shape sized and shaped to substantially fit only over a posterior superior portion of a user's head;
engagement means for removably securing an object to said support base; and
means for connecting said support base to a goggle strap.

25. The aquatic device as claimed in claim 24 wherein said engagement means further includes at least one fastener.

26. The aquatic device as claimed in claim 25 wherein said fastener includes a hook and loop fastener.

27. The aquatic device as claimed in claim 25 wherein said fastener includes a magnetic fastener.

28. The aquatic device as claimed in claim 25 wherein said fastener includes a channel and a pin sized and shaped to fit within said channel.

29. The aquatic device as claimed in claim 28 wherein said channel is disposed on an exterior surface of said support base, said channel having a first and a second end disposed on opposites ends of said channel wherein said first end is substantially open and said second end includes a flanged region.

30. The aquatic device as claimed in claim 29 further including a biased tab disposed proximate sand first end, said biased tab urging said object towards said flange region of said channel.

31. A sport apparatus comprising:
a goggle;
a strap secured to said goggle;
a support base secured to said strap, said support base sized and shaped to substantially fit only over a posterior superior portion of a user's head; and
engagement means for removably securing an object to said support base.

32. The sport apparatus as claimed in claim 31 wherein said object includes a snorkel.

33. The sport apparatus as claimed in claim 31 wherein said object includes a light.
